(19) United States
(12) Patent Application Publication
(10) Pub. No.: US 2025/0295593 A1
(43) Pub. Date: Sep. 25, 2025

(54) FUNCTIONALLY-DIVIDABLE ORODISPERSIVE DOSAGE FORM

(71) Applicant: APRECIA PHARMACEUTICALS LLC, Mason, OH (US)

(72) Inventors: Lauren E. BEACH-HERRERA, East Brunswick, NJ (US); Kelly E. CAPUTO, Langhorne, PA (US); Jaedeok YOO, Princeton, NJ (US); Thomas J. BRADBURY, Yardley, PA (US)

(21) Appl. No.: 19/210,666

(22) Filed: May 16, 2025

Related U.S. Application Data
(63) Continuation of application No. PCT/US2023/080212, filed on Nov. 17, 2023.
(60) Provisional application No. 63/426,690, filed on Nov. 18, 2022.
(60) Provisional application No. 63/592,206, filed on Oct. 22, 2023.

(51) Int. Cl. A61K 9/20

(52) U.S. Cl. CPC A61K 9/2072; A61K 9/2095

(57) ABSTRACT

A dividable tablet with a functional seam for functionally dividing the dividable tablet into substantially equivalent sub-dosage units along the functional seam. The functional seam is configured to subdivide the sub-dosage units into the same mass within a tolerance of about 3% or less, and typically of 1.0% or less. The functional seam is configured reduce the amount of lost material that separates from the divided sub-dosage units after division of the dividable tablet along the functional seam, to less than 3%, and typically less than 1.0%, and more typically less than 0.1% of the mass of the undivided dividable tablet.

### CROSS-REFERENCE TO EARLIER FILED APPLICATIONS

The present application is a continuation of International Application No. PCT/US2023/080212 filed Nov. 17, 2023, which claims the benefit of U.S. Provisional Application No. 63/426,690 filed Nov. 18, 2022, and U.S. Provisional Application No. 63/592,206 filed Oct. 22, 2023, the entire disclosures of which are hereby incorporated by reference.

### FIELD OF THE INVENTION

The present invention relates in general to a dosage form or a tablet that can be subdivided into two or more sub-dosage units.

### BACKGROUND OF THE INVENTION

It is well known in the pharmaceutical art that dosage forms such as orally administered tablets may be formed with one or more grooves or physical score lines to facilitate breakage of the tablet into individual fragments or sub-dosage units. This can allow a pharmaceutical composition to be provided to patients in measured, predetermined doses. Each sub-portion can generally include a proportionally equal fractional amount of the prescribed pharmaceutical dosage, with tablets often being dividable into half portions, third portions, or quarter portions. Being able to divide tablets in such a manner allows for dose flexibility, cost mitigation, and can help with ease of administration in pediatric and geriatric patients who have difficulty swallowing large tablets.

For many years the pharmaceutical industry has been called upon to improve the quality of tablet breaking. The problems with conventionally scored tablets include unequal breaking and/or loss of mass after division, which can lead to over or under dosing. Ultimately the problem of breaking conventionally scored tablets can reside in the hardness of the tablet resulting from the compression techniques used to form the tablet. Further, the small size configuration of the tablet may not allow for easy breakage. In fact, a sharp instrument is often required to sever a conventionally scored tablet, which frequently results in breakage of the tablet into undesired miniature pieces of inaccurate dosages.

A need remains for an improved dividable tablet, and in particular a three-dimensionally-printed dividable tablet form.

### SUMMARY OF THE INVENTION

The present invention provides a dividable tablet including one or more functional seams for functionally dividing the dividable tablet into two or more sub-dosage units, including three, four or more sub-dosage units, along the one or more functional seam.

In some embodiments, the two, three, or more sub-dosage units can be the same volumetric size; in other embodiments, of different volumetric sizes. In some embodiments, after the dividable tablet has been divided, the divided sub-dosage units have the same mass with a mass difference between the two sub-dosage units being about 3% or less, and typically of 2.0% or less, 1.5% or less, and more typically of 1.0% or less, and 0.5% or less.

In some embodiments, the functional seam comprises a planar segment of the tablet, including one or more planar segments of the tablet, which extends through the entire volume of the tablet. The planar segment extends along the two dimensions of a plane.

In some embodiments, the functional seam can comprise a curved segment of the tablet, including one or more curved segments of the tablet, which extends through the entire volume of the tablet. A curved segment extends along the three dimensions of a plane curve of a slight curvature, which typically has, for a circular tablet, a radius of curvature of greater than a radius of the circular tablet, including greater than a diameter of the circular tablet, and for an other-shaped tablet, including an oblong or triangular-shaped tablet, a radius of curvature of greater than a minimum dimension of the other-shaped tablet, including greater than a maximum dimension of the other-shaped tablet.

In some embodiments, a dividable tablet can comprise two or more functional seams selected from one or more planar segments, one or more curved segments, or a combination thereof.

In some embodiments, the amount of material of the dividable tablet that separates or is lost from the resulting two or more sub-dosage units, after the dividable tablet has been divided, is less than 3%, and typically less than 1.0%, and more typically less than 0.1% of the mass of the undivided tablet.

A dividable tablet of the present invention can be a three-dimensionally-printed (3DP) dividable tablet.

In various embodiments, each sub-dosage unit of a dividable tablet includes a breakage-resistant boundary wall joined to and along an opposite side of the one or more functional seam.

In some embodiments, the breakage-resistant boundary wall is a peripheral segment of the sub-dosage unit that is joined to a side of the functional seam, and accordingly, the peripheral segment of the sub-dosage unit can be a planar breakage-resistant segment that is joined to and along a side of a planar functional seam; or can be a curved breakage-resistant segment that is joined to and along a side of a curved functional seam.

A functional seam can comprise a first bound powder matrix having a first porosity, and the boundary wall of each sub-dosage unit comprises a second bound powder matrix having a second porosity that is less than the first porosity. In some embodiments, the first bound powder matrix of the functional seam has a uniform porosity along the planar structure of the functional seam, and in some embodiments, the second bound powder matrix of the boundary wall has a uniform porosity along the entire plane of the boundary wall. In some embodiments, the porosity or the uniform porosity of the first bound powder matrix of the functional seam is higher than, and typically at least 10% higher, and more typically at least 15% or at least 25% higher, than the porosity or the uniform porosity of the second bound powder matrix of the boundary wall. The first bound powder matrix of the functional seam having the higher porosity comprises at least 50%, and preferably at least 90%, and up to 100% of the planar structure of the functional seam.

The 3DP dividable tablet can be subdivided into multiple sub-dosage units, such as two, three, four or more sub-dosage units. In various embodiments, the dividable tablet, and the sub-dosage units of the tablet, is ingestible and orodispersible, and can be rapidly orodispersible. In some embodiments, the dividable tablet can include a single functional seam through the center of the tablet that, when divided or broken, divides the tablet into two equal sub-dosage units.

In various embodiments, a dividable tablet comprises a functional seam having a resistance to a bending force that is less than a resistance to the bending force of the at least two boundary walls.

Without being bound by any particular theory or mechanism, applying a bending force onto the sub-dosage units on opposite sides of the functional seam will cause a failure in bending of the dividable tablet, where the bending moment of the sub-dosage units generate tensile stresses that exceed the yield stress of the functional seam, subdividing the dividable tablet along the functional seam into separate sub-dosage units, without a failure in bending or a breaking of the boundary walls of the sub-dosage units.

An embodiment of the invention provides a three-dimensionally printed tablet including: (a) at least two sub-dosage units, each of the at least two sub-dosage units comprising a body including a planar boundary wall, wherein the planar boundary walls of the at least two sub-dosage units are oriented in parallel and confront one another, and (b) a functional seam positioned between and joining the confronting planar boundary walls of the at least two sub-dosage units, wherein the functional seam comprises a first bound powder matrix having a first resistance to a bending force, and the respective planar boundary walls comprise a second bound powder matrix having a second resistance to the bending force, wherein the second resistance of the planar boundary walls to the bending force is higher than the first resistance of the functional seam to the bending force. Each planar boundary wall resists breakage by the bending force that breaks the functional seam, so that the tablet can be divided broken along the functional seam, including under ordinary handling conditions. The planar boundary walls of the at least two sub-dosage units resist breakage and typically do not break, and as compared to the functional seam, the planar boundary walls provide a controlled breakage and division of the tablet along the functional seam into the two (or more) sub-dosage units.

In various embodiments, the second resistance of the boundary walls to the bending force is at least 10% higher, more typically twice as high, and even more typically several times higher, than the first resistance of the functional seam to the bending force.

In various embodiments, a dividable tablet can comprise an active pharmaceutical ingredient (API). In some embodiments, the dividable tablet can comprise from 0.1% to 80% by weight API. In some of the same or different embodiments, the dividable tablet can deliver an API dosage amount of at least 5 micrograms (μg) and up to 5,000 milligram (mg), including at least 10 μg, and up to 2,500 μg.

In some embodiments, the tablet includes a total, predetermined mass (or volume or other unit) amount of the API and can be subdivided along the functional seam so that each sub-dosage unit contains a sub-dose mass amount of the API that is a pre-determined fraction or portion of the total amount of API. In some embodiments, each sub-dose mass amount of the API is equal in mass. In some other embodiments, the respective sub-dose mass of the API can be different; for example, the sub-dose mass of the API in a first sub-dosage unit can be some percentage greater than, or a multiple of, the sub-dose mass amount of the API in a second sub-dosage unit.

Another embodiment of the invention, a body of the at least two sub-dosage units includes a boundary wall, an outer periphery wall, and an interior body portion. The interior body portion can comprise a powder material or a bound powder matrix that is different from the bound powder matrices of the boundary wall and the outer periphery wall.

The invention also provides a method of making a 3DP dividable tablet, comprising the steps of providing a plurality of layers of a powder, and printing one, more or all of the plurality of layers of the build powder with a printing pattern of a binding liquid, wherein the printing pattern includes one or more printing gaps that divide the printing pattern into at least two sub-dosage printing patterns, wherein the printing gap has a reduced binder saturation, or no binder saturation, as compared to a boundary printing pattern of each sub-dosage printing patterns along the opposite sides of the printing gap, thereby forming a plurality of bound-powder layers of the 3DP tablet. Preferably, the printing gap is an area where no binding liquid is printed. In some embodiments, the printing gap provides a corresponding printing of binding liquid having a width, between the at least two printed sub-dosage units, of at least about 100 microns (μm), and typically up to about 500 μm. Preferably, the printing pattern of the boundary wall of each at least two sub-dosage printing pattern, has a print width, extending from the printing gap and transverse to the axis of the printing gap, which provides a corresponding printing gap in the printing of binding liquid having a width of at least about 100 μm, including at least about 200 μm, at least about 300 μm, and preferably at least about 500 μm. Each bound-powder layer comprises a first bound-powder boundary region, a second bound-powder boundary region, and a functional seam bound-powder region. The first bound-powder boundary region is formed by a boundary pattern portion of a first sub-dosage printing pattern, and the second bound-powder region is formed by a boundary pattern portion of a second sub-dosage printing pattern. The seam bound-powder region is joined to and between the first and second bound-powder boundary regions, and the seam bound-powder region has a lower resistance to a bending force than a resistance to the bending force of a bound powder matrix of both the first and second bound-powder boundary regions.

In any of the various embodiments, the dividable tablet does not include a physical score line, such as a groove or gulley, in an outer surface of the tablet, along an axis of the functional seam.

In any of the various embodiments, the tablet can include a visible marking on or visible through the outer surface of the functional seam to facilitate the identification of the functional seam and the positioning of the tablet for sub-dividing along the functional seam.

In various embodiments, for any of the dividable tablets described herein, the dividable tablets and respective divided sub-dosage units comply with some or all of the criteria for functional scoring described in U.S. FDA's Guidance for Industry—Tablet Scoring: Nomenclature, Labeling, and Data for Evaluation March 2013, hereby incorporated by reference in its entirety. In various embodiments, for any of the dividable tablets described herein, the tablet sub-dosage units optionally can demonstrate adequate stability for a period of 90 days at 25° C., plus or minus 2° C. and 60 percent Relative Humidity (RH), plus or minus 5 percent RH, when stored in pharmacy dispensing containers (no seal/no desiccant). In various embodiments, for any of the dividable tablets described herein, the tablet sub-dosage units optionally can meet the same finished-product testing requirements as for a whole-tablet product with equivalent strength when split non-mechanically (by hand) and mechanically (with a tablet splitter). In various embodiments, for any of the dividable tablets described herein, the divided sub-dosage units optionally meet finished product release requirements for disintegration.

The invention includes combinations of the aspects, embodiments and sub-embodiments of the invention disclosed herein.

### BRIEF DESCRIPTION OF THE FIGURES

The accompanying drawings illustrate the prior art and preferred embodiments of the invention and, together with a general description of the invention given above, and the detailed description given below, explain the principles of the invention.

FIG. 1 illustrates a perspective view of an incrementally layered prior art tablet constructed from a three-dimensionally printed, bound powder matrix.

FIG. 2 illustrates a cross-sectional view of the tablet of FIG. 1, taken along line 2-2 of FIG. 1.

FIG. 3 illustrates a printing pattern used to apply binding liquid to a base layer and top layer of build powder, for the tablet illustrated in FIG. 1.

FIG. 4 illustrates a printing pattern used to apply binding liquid to intermediate incremental layers of build powder, for the tablet illustrated in FIG. 1.

FIG. 5 illustrates a top perspective view of a dividable tablet having a functional seam that extends along a dissecting line and joins or bonds together two sub-dosage units.

FIG. 6 illustrates the dividable tablet of FIG. 5 as a series of incremental, bonded layers of bound powder.

FIG. 7 illustrates a perspective view of the dividable tablet of FIG. 5 with an irregular segment removed from the sub-dosage units along the functional seam through line 7-7 of FIG. 5.

FIG. 8 illustrates a first printing pattern of the binding liquid used to print the top and bottom layers of the dividable tablet of FIG. 5.

FIG. 9 illustrates a second printing pattern of the binding liquid used to print the plurality of intermediate layers of the dividable tablet of FIG. 5.

FIG. 10 illustrates the dividable tablet of FIG. 5 that has been divided or broken along the functional seam, dividing the tablet into two, typically equal, sub-dosage units.

FIG. 11 illustrates another embodiment of a dividable tablet having a physical score line formed within the top layer of bound powder.

FIG. 12 illustrates a printing pattern used in printing of the top layer of bound powder illustrated in FIG. 11.

FIG. 13 illustrates a plan view a computed topographic (CT) scan taken laterally through the height of the dividable tablet, parallel with the incremental bound-powder layers.

FIG. 14 shows the CT scan of FIG. 13, with the boundaries between the functional seam, boundary walls, peripheral walls, and interior portions illustrated in dashed lines to better distinguish such elements.

### DETAILED DESCRIPTION OF THE INVENTION

### Definitions

As used herein, an “equivalent sub-dosage units” means separated sub-dosage units of a tablet that have substantially equal mass, and preferably equal shape and volume.

As used herein “break strength” is a measure of an unreinforced tablet to resist failure in bending under a force applied along a lateral portion of the tablet, and typically a lateral centerline of the tablet.

As used herein, “ordinary handling conditions” means the typical conditions of manufacturing, processing, packaging, transportation and storage to which tablet products of such kind are typically exposed in industry.

### Bound Powder Matrix

The tablet can be generally made of a bound powder matrix preferably, though not necessarily, containing a pre-determined total mass (or volume) amount of an active pharmaceutical ingredient (API). The dosage forms are formed using additive manufacturing or three-dimensional-printing (3DP) techniques, particularly binder-jetting processes, to form a porous, bound powder matrix having a fixed three-dimensional shape. Methods for using binder-jetting processes to construct a bound powder matrix from a build powder and a binding liquid are described in further detail below. The bound powder matrices are rapidly orodispersible because they undergo immediate and very rapid disintegration when placed in a small volume of aqueous fluid, such as water, saliva, juice, milk, beverage, body fluid, soda, or a combination thereof.

In some embodiments, the tablet can disintegrate and its particulate components can disperse within a minute, such as within about 30 seconds, or within about 15 seconds, or within about 5 seconds, or within about 1 second, when placed in a small volume (for example, in 25 ml or less, or in 10 ml or less, or in 5 ml or less, or 2 ml or less, or 1 ml or less) of water or saliva, thereby facilitating easy swallowing and administration.

Three-dimensional printing (3DP) of pharmaceutical dosage forms is generally known, and can include a solid freeform fabrication technique/rapid-prototyping technique in which a thin layer of a build powder, including a powder containing a uniform concentration of an API, are spread onto a surface, and selected regions of the thin layer of powder are bound together by controlled deposition (“printing”) of a binding liquid. This basic operation is repeated incrementally, adding layer-by-layer of build powder, with each new layer being formed on top of, and adhered to, the previously printed layer, to eventually make a unitary tablet. In various embodiments, the unitary tablet is a porous tablet, comprising particles of solid ingredients, typically of a powder, which are bound together into a porous, bound powder matrix.

In various embodiments, a plurality of tablets can be formed within an open powder bed, where a binding liquid is selectively printed in print areas onto incremental layers of powder material to form incremental, bound-powder layers of a unitary tablet, leaving unprinted the remaining powder of the open powder bed. After all incremental layers of the tablets have been printed, and the bound-powder layers of the printed tablets have sufficient cohesion, the printed tablets are separated from the unprinted, unbound powder, dried, dedusted, and packaged. In another embodiment, a tablet can be formed within a dedicated cavity in the shape of the finished tablet. A particularly suitable printing assembly for three-dimensional printing of the unprinted, sub-dividable dosage forms described herein can include build modules, a powder layering system, a printing system, a printing liquid removal (or drying) system, and a dosage form handling system. Various systems and methods for making dosage forms are described in U.S. Pat. Nos. 8,888,480, 9,517,591, 9,517,592, 9,610,735, 9,908,293, 10,118,335, 10,449,712, 11,097,483, and 11,278,501, and U.S. Published Appl. No. 2018/0141275, all of which are incorporated herein by reference in their entireties.

### Binding Liquid Printing

Three-dimensional printing of binding liquids can have spatial descriptors in each of three different, typically orthogonal directions. In three-dimensional printing, binding liquid may be dispensed from a single print port of a print nozzle in droplets or in liquid units resembling droplets. A layer of powder material can be moved in a horizontal plane directly beneath a print port, in a longitudinal direction of motion. The single print port can be moved laterally relative to a surface of the layer of the powder material while dispensing droplets vertically from the single print port in a succession that deposit onto the surface of the layer of powder material, also referred to herein as a layer of build powder, to form a line of droplets corresponding to the line of movement of the printhead over the layer of build powder. The spacing between the successive droplets deposited in the lateral line on the surface of the build powder layer is referred to as a drop-to-drop spacing, or droplet spacing. After completion of one lateral line of droplets along the surface of the layer of build powder, the layer of build powder is repositioned in a longitudinal direction relative to the printhead, and another lateral line of droplets is deposited adjacent to the previously-deposited line of droplets, and separated longitudinally therefrom by a distance referred to as a line-to-line spacing. After completion of printing on a layer of powder, another powder layer may be deposited, with each powder layer having a layer thickness. The powder layer thickness is the third descriptor.

In the printing of binding liquid onto a layer of build powder, the spacing of droplets from a single print port deposited along the moving surface of the layer of build powder moving below the print nozzle may be described in terms of the resolution of the printing system, often expressed as dots per inch (dpi), where a dot is a single droplet and dpi is the reciprocal of droplet spacing. For example, resolutions of 300 and 600 dpi correspond to droplet spacings of about 84.7 microns and about 42.3 microns, respectively. The droplet spacing (within a line), or the line spacing (spacing of droplets from one line to the next), or any other spacing of droplets may be described in terms of resolution expressed in dpi. One can determine the number of droplets applied to a specified area of the top surface of a powder layer by the droplet spacing and the line spacing, and conversely, given the required number of droplets required on a specific area, a droplet spacing and line spacing can be determined.

In various embodiments, the print nozzle comprises a series of print ports in a row or linear series. Typically, the series of print ports will be arranged laterally, transverse to the longitudinal direction of relative movement of the layer of build powder in the horizontal plane. Generally, a layer of powder will move horizontally in the longitudinal (machine) direction under an array of print ports. The binding liquid dispensed from each port of the lateral series of print ports forms a longitudinal line of successive droplet deposits onto the layer of build powder moving beneath the print ports. The frequency of droplets dispensed from a print port is described as droplets per second (hertz, or Hz), and depending on the velocity of the layer of build powder moving longitudinally beneath the print port, the deposits of printing liquid along the longitudinal line are described as droplets per unit length, such as droplets per inch or droplets per centimeter.

The distance between adjacent print ports defines the lateral distance between the longitudinal lines of successive droplet deposits, which is defined as the line-to-line spacing, or line spacing, which is the reciprocal of the number of print ports per unit of length, such as per inch or per centimeter.

Another droplet factor is the volume or corresponding diameter, and the corresponding mass, of the droplets. Controlling the size, and the corresponding mass, of the droplets for a selected droplet resolution allows control of the total mass of binding liquid applied to the specified area of the top surface of a powder layer.

In the printing of binding liquid onto layers of build powder to form one or more printed article, a print pattern is generated for each incremental layer of build powder that forms the printed article. The print pattern instructs the print nozzle on the timing, size (mass quantity) and frequency of the droplets to be dispensed from selected ports of the print nozzle to achieve both the shape of the print pattern of droplets and the mass or volume of the binding liquid deposited onto each areal portion of the layer of build powder corresponding to the print pattern.

In the printing of binding liquid onto a single layer of build powder, the print pattern can be represented by a pixelated image characterized by a resolution in dots-per-length (for example, dots-per-inch) in each of two orthogonal linear directions, each dot corresponding to a droplet. In some instances, these pixelated images are 1-bit monochrome images, alternately referred to as binary or bi-level images in which each pixel contains one bit of information (0 or 1) that may be represented as either black or white onscreen. A pattern in which all pixels are black is referred to as a “solid” printing pattern. For making dosage forms from a series of layers of build powder, a series of pixelated images is prepared and used, representing the layer-by-layer printing instructions for each succussive layer of build powder for the printing of the article.

In some instances, an amount of binding liquid applied in a localized areal region(s) of the dosage form can be achieved by using a combination of solid printing and “grayscale” printing, which uses a grayscale print pattern in the dosage form design. In the case of 1-bit monochrome images used for machine instructions, grayscaling is achieved by changing the number of “black” pixels relative to “white” pixels in a chosen region of a dosage form, or in a chosen layer of a dosage form, or throughout a dosage form. Other regions may be “solid” by using all black pixels. In some embodiments, the dosage form design includes a “solid” exterior or periphery, and a “grayscaled” interior, which results in fewer droplets of liquid per unit area within the interior of the printed region as compared to the area of the outer periphery. In some embodiments, grayscaling may be achieved with equally spaced black pixels amongst white pixels to reach an overall ratio of black to white pixels in the grayscaled region. In other embodiments, grayscaling may be achieved with randomly placed black pixels amongst white pixels to achieve an overall ratio of black to white pixels in the grayscaled region. In still other embodiments, grayscaling may be achieved with a chosen pattern (e.g., parallel lines, hashed pattern, dot pattern) of black pixels amongst white pixels to achieve an overall ratio of black to white pixels in the grayscaled region.

In some embodiments for the printing of binding liquid onto a layer of build powder to form one or more printed article, the saturation level of binding liquid applied to a selected surface area(s) or region(s) of a powder layer surface can be expressed as an applied quantity of the binding liquid within a selected area of the powder layer to be wetted. To account for the depth of a powder layer, and thus the volume of powder being wetted by a quantity of the binding liquid, the saturation level can be defined as the volume or mass, for example milligrams (mg), of binding liquid applied per unit of surface area of the powder layer, for example square centimeters (cm²), per thickness of the powder layer, and typically the average thickness of the powder layer, for example millimeters (mm). In various embodiments, a saturation level of a binding liquid onto a layer of powder is about 1 to 60 mg/cm²/mm, for example, about 4 to 32 mg/cm²/mm. The specific saturation of a binding liquid will vary depending, without limitation, upon the components of the binding liquid, the components of the powder material that forms the powder layer, the size distribution of the particles of the powder material, and the desired properties of the bound powder matrix of the printed tablet.

A full saturation level of binding liquid is a quantity, including either a mass quantity or a volumetric quantity, of binding liquid applied as droplets to a unit area of a layer of powder material per unit depth of the layer, which is sufficient to migrate downwardly through the powder material within the unit area to a full depth of the layer of powder material, and to wet the powder material within the print area sufficiently to bond the particles of the powder material together into a unitary bound powder matrix that has a tenacity sufficient to avoid breaking thereof under ordinary handling conditions, though without overly or excessively saturating and dissolving most or all of the water-soluble components of the powder material. The quantity of binding liquid deposited onto the layer of powder material will typically migrate into the powder material surrounding the area where the liquid was deposited, and dissolve some amount of any dissolvable compound within the powder material that is contacted. Excessively low saturation levels tend to result in poor structural integrity of the resulting bound powder material. Exceedingly high saturation levels tend to result in excessive bleeding of liquid beyond where the liquid was deposited and intended to flow, and potentially excessive dissolving of compounds of the powder material into the binding liquid. A person of ordinary skill in the art, for a particular powder material and binding liquid, will be able to determine and define a full saturation value of a binding liquid with minimal testing.

A full or 100% saturation level hereinafter can then be determined and designated for a particular powder material and binding liquid. In some embodiments, a full or 100% saturation level is about 8 to 60 mg/cm²/mm of binding liquid, including 10 to 40 mg/cm²/mm, and more typically about 12 to 32 mg/cm²/mm. Generally, use of more than full or 100% binder saturation is not needed to provide effective bonding of the powder material, and can unnecessarily increase the drying requirements to evaporate excess free binding liquid.

Having determined a 100% saturation, a binding liquid can be applied onto a particular area of the powder material layer in a quantity of 100% saturation, or more, or less than 100% saturation. For example, a binding liquid can be applied onto a particular area of the powder material layer in a quantity less than 100% saturation, such as to 90%, or 80%, or 70%, or 60%, or 50%, or 40%, or 30%, or 20%, or 10% saturation, or saturations therebetween. In general, saturation levels less than full or 100% saturation for a particular powder material and binding liquid are also referred herein as “grayscale” saturations.

In various embodiments, the quantity of binding liquid within a selected area of a powder layer is about 0.6 mg/cm²/mm to about 60 mg/cm²/mm, and more preferably about 6.2 mg/cm²/mm to about 40.0 mg/cm²/mm. In various embodiments, a level of binding liquid to be applied to at least the peripheral portions (sidewalls, and top and bottom surfaces) of the completed tablet is typically about 25.0 to about 35.0 mg/cm²/mm, and for example, about 30.0 to about 32.0 mg/cm²/mm, and is sufficient to provide structural integrity to the finished tablet during handling.

Suitable printing devices include those having a continuous inkjet printer (CIJ) or those having a drop-on-demand printhead. A continuous jet printhead provides a continuous jet (spray) of droplets that can be selectively charged while passing through plates and targeted onto a powder layer, while uncharged droplets are not deflected and return to the CIJ system. A drop-on-demand printhead only deposits droplets of printing fluid onto the powder layer if it receives an instruction (demand, operational command) to do so. A printhead scans (applies fluid to) the surface of a powder layer at a predetermined rate, e.g. a scan rate, to form a line of droplets. A high scan rate will result in a lower saturation level, and a low scan rate will result in a higher saturation level when comparing printing fluid deposition at a constant volume per unit time. An increase in the scan rate from 1.0 m/s to 2.0 m/s reduces the total volume of binder solution deposited in the tablets by half. As the print speed increases, the apparent density of the printed article (theoretical, calculated from the weight and dimensions of the tablet) decreases. A simultaneous decrease in the dimensions and weight of the tablets is also seen. This decrease is attributed to the fact that a decrease in the total volume of binder droplets deposited onto the powder results in a decrease in the extent of binder solution spreading in the powder. Increasing the print speed also decreases the flash time and the hardness and increases the friability of the tablets. This result is obtained because the proportion of binding liquid decreases in the tablets as the print speed increases. An increase in the print speed also increases the void volume inside the tablets, as illustrated by an increase in the percent volume of the tablets penetrated by mercury at 30 psi (% intrusion).

When using a continuous jet printhead, the printhead scans at a rate of about 0.5 to 3.0 m/sec, and most preferably at about 1.75 m/sec. When using a drop-on-demand jet printhead, the printhead scans at a rate of 0.1 to 1 m/sec, most preferably at about 0.15 to about 0.5 m/sec.

The volume of individual droplets can be varied as desired, for example, by selection of a different three-dimensional printing machine, or different printhead components on the same machine, or different parameters on the same printhead and same machine. Increasing the volume of the droplet increases the saturation level and decreasing the volume of a droplet decreases the saturation level when comparing printing fluid deposition at a constant scan rate. When using a continuous jet printhead, the size of the fluid droplets delivered by the printhead preferably ranges from about 15 μm to about 150 μm in diameter. When using a drop-on-demand printhead, the size of the fluid droplets delivered by the printhead preferably ranges from about 40 μm to about 60 μm in diameter.

The flow rate of the liquid delivered by the printhead can be varied as desired. Increasing the flow rate will increase the saturation level by increasing the droplet size and/or droplet resolution. Decreasing the flow rate decreases the saturation level when comparing printing fluid deposition at a constant scan rate. As discussed herein, the printhead deposits droplets of printing fluid to form parallel lines thereof in the powder layer. When using a continuous jet printhead, the line spacing ranges from about 20 to about 1000 μm, or about 50 to about 500 μm, and preferably about 100 to 200 μm. When using a drop-on-demand jet printhead, the line spacing ranges from about 20 to about 300 μm, about 40 to about 100 μm, or about 55 to 75 μm.

The instructions for printing of the print pattern take into account parameters of the build powder and the binding liquid, and the movement velocity of the layer of build powder relative to the printhead and its ports. The parameters of the build powder can include the mass portion and hydroscopicity of the various build powder components. In general, water-absorbing or water-soluble powder materials will take up more binding liquid than a water-repelling or water-insoluble material, while requiring less binding liquid to form a wetted bound-powder matrix that, once dried by removing excess water and solvent, forms a stable, hard and non-friable bound-powder matrix. The parameters of the binding liquid can include the mass portion of water and of solvents, and the mass portion of any dissolved binder components used in the binding liquid.

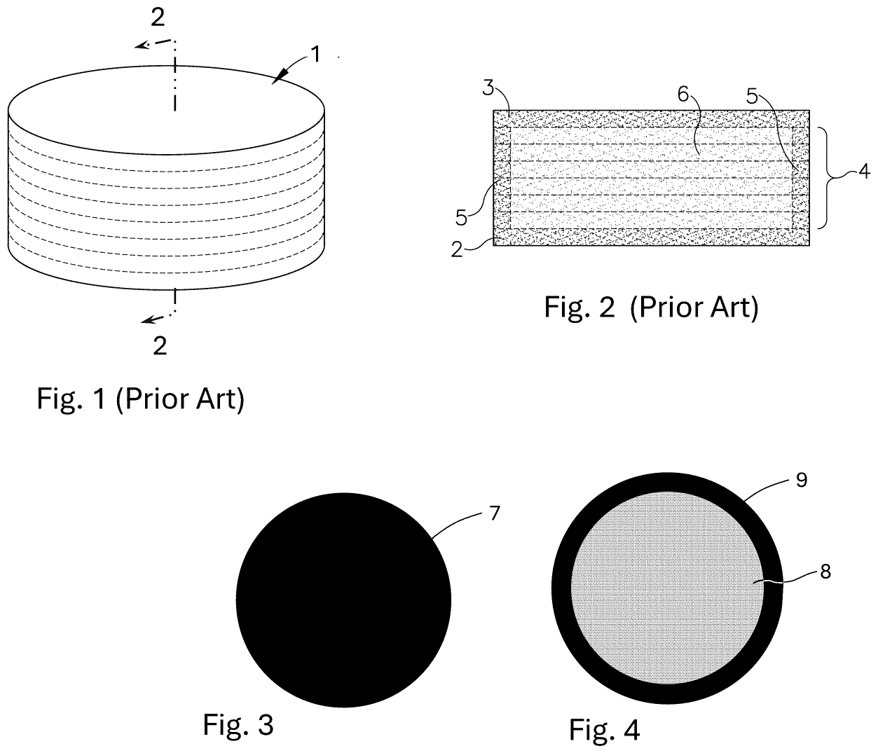

FIG. 1 is a perspective view of a prior art orodispersible tablet 1 made from a three-dimensionally printed (3DP) matrix of a bound powder. FIG. 2 is a cross-sectional view of the prior art orodispersible tablet 1 comprising sequentially formed incremental layers of bound powder, having a base layer 2, a top layer 3, and one or more intermediate layers 4. The 3DP tablet is formed by a binding liquid that is printed or applied onto a surface of a powder layer to form the bound powder matrix. The exterior surface portions of the tablet 1 include the base layer 2, the top layer 3, and the peripheries 5 of the plurality of intermediate layers 4. The interior of the tablet 1 is formed by the interior portions 6 (inboard of the periphery 5) of the plurality of intermediate layers 4. The 3DP matrix structure forming the exterior surfaces of the tablet 1 has a break strength that can be higher (in resistance to a breaking force) than a break strength of the interior portions 6. The higher break strength of the exterior surfaces of the tablet 1 envelopes and contains the interior portions 6, to provide structural integrity and breakage resistance, while both the exterior surface portions and the interior portions 6 provide sufficient orodispersibility.

In various embodiments, the higher hardness of the exterior surface portions can be achieved by applying a higher saturation level (for example, by using a higher mass of a binding liquid applied onto the powder layer in that region, by applying a binding liquid containing a higher content of the same or different binder, or a combination thereof) as compared to the interior portions. If the conventional tablet 1 shown in FIG. 1 were to be bent over a fulcrum along the centerline of the bottom of the tablet, it is possible that the tablet would break into two or more pieces, and the interior portions 6 of the intermediate layers 4 would be exposed. It would be possible that the tablet will not break along the centerline into two equal or functionally-equivalent parts. It is also possible that the powder matrix in the interior portions 4 may fall away from the bound powder matrix of the interior portions 4, resulting in a loss of the powder material within the interior portions 4. This would be problematic for a medicament or pharmaceutical tablet or dosage form, especially if an active pharmaceutical ingredient or API were contained within the ingredient powder of the bound powder matrix.

FIGS. 3 and 4 show plan views of liquid printing patterns used to apply binding liquid on the layers of powder to form incrementally printed layers of the tablet 1 shown in FIGS. 1 and 2.

FIG. 3 depicts a solid printing pattern 7 used to create the base layer 2 and the top layer 3 of tablet 1, wherein a level of binding liquid is evenly applied throughout the entire printing area. The droplets can be dispensed at deposition points (dots) at a fixed density in a nozzle direction (for example, a density of nozzles spaced apart 400 dots per inch (dpi) on the print head) and at some predefined and selectively variable density in an operation direction, depending upon the relative speed of the powder layer and the print head. In some embodiments, the quantity of liquid dispensed by a nozzle at a dot is constant, such that the density of the dots in the operation direction determines the quantity of liquid that a particular powder material will receive from the print head per unit area of powder. In some embodiments, a predefined and selectively variable density in the operation direction can range from about 600 to 1800 dpi.

The specific saturation of a binding liquid will vary depending upon the components of the binding liquid, the components of the powder material that forms the powder layer, the size distribution of the particles of the powder material, and the desired properties of the bound powder matrix of the printed tablet.

A full saturation level of binding liquid is a quantity, including either a mass quantity or a volumetric quantity, of binding liquid applied as droplets to a unit area of a layer of powder material per unit depth of the layer, which is sufficient to migrate downwardly through the powder material within the unit area to a full depth of the layer of powder material, and to wet the powder material within the print area sufficiently to bond the particles of the powder material together into a unitary bound powder matrix that has a tenacity sufficient to avoid breaking thereof under ordinary handling conditions, though without overly or excessively saturating and dissolving most or all of the water-soluble components of the powder material.

A person of ordinary skill in the art, for a particular powder material and binding liquid, will be able to determine and define a full saturation value of a binding liquid with minimal testing. A full saturation level hereinafter can then be designated as a 100% saturation level for a particular powder material and binding liquid. In some embodiments, a full or 100% saturation level is about 8 to 60 mg/cm²/mm of binding liquid, more preferably about 12 to 32 mg/cm²/mm. Ideally, use of more than full or 100% binder saturation is not needed to provide effective bonding of the powder material, and will unnecessarily increase the drying requirements to evaporate excess free binding liquid.

Having determined a 100% saturation, a binding liquid can be applied onto a particular area of the powder material layer in a quantity of or less than 100% saturation. For example a binding liquid can be applied onto a particular area of the powder material layer in a quantity less than 100% saturation, such as to 90%, or 80%, or 70%, or 60%, or 50%, or 40%, or 30%, or 20%, or 10% saturation, or saturations therebetween. In general, as referred to herein, saturation levels between less than full or 100% for a particular powder material and binding liquid are referred to as “grayscale” saturations.

FIG. 4 shows a second liquid printing pattern that includes two distinct printing regions, including an interior portion 8 and a peripheral portion 9. In the illustrated embodiment, the interior portion 8 of the printing pattern is a substantially uniform grayscale saturation level. The peripheral portion 9 of the printing pattern comprises a substantially uniform, full saturation level of binding liquid used only in an outer ring of the printing area. The printing pattern shown in FIG. 4 is used to create the plurality of intermediate layers 4 of tablet 1.

In some embodiments, a solid printing pattern used to create a base layer, a top layer, and the peripheral portions of the intermediate bound-powder layers can have a grayscale saturation level (less than 100% saturation), and for example more than 50% and up to 100% saturation level, provided that such saturation level of binding liquid dispensed on the powder material forms a bound powder matrix that creates a unitary bound-powder tablet that can be made, handled, packaged, storage, distributed and used by a user, under normal conditions, without breaking or cracking, or losing a substantial portion of its powder material.

Even though these printing patterns are depicted as circular, any geometry can be used, e.g. oval, square, rectangle, oblong, polygonal, and others.

### Dividable Tablets

If a conventional tablet 1 such as one shown in FIG. 1 is struck with a utensil along the centerline of the top of the tablet, the conventional tablet possibly would break into two or more pieces, and the interior portions 6 of the intermediate layers 4 would be exposed. It would be possible that the tablet will not break along the centerline into two equal or functionally equivalent parts. It is also possible that the powder matrix in the interior portions 4 (FIG. 2) may fall away from the bound powder matrix of the interior portions 4, resulting in a loss of the powder material within the interior portions 4. This would be problematic for a medicament or pharmaceutical tablet or dosage form, especially if droxidopa were contained within the ingredient powder of the bound powder matrix.

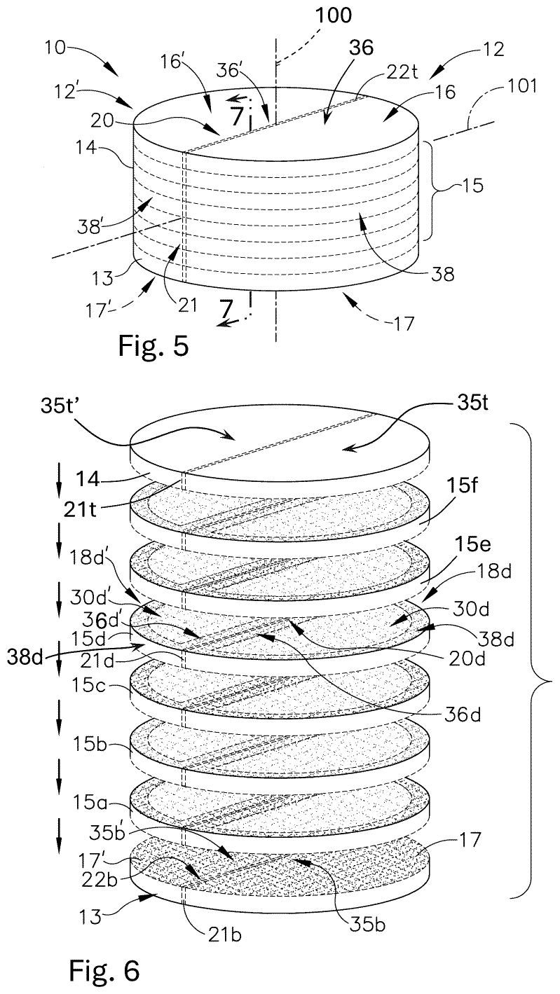

A dividable tablet 10 is illustrated in FIGS. 5-7. FIG. 5 illustrates a top perspective view of the dividable tablet 10 having a functional seam 20 comprising, and typically consisting of, a bound powder matrix that extends along a bisecting line 101 and joins or bonds together two opposing planar, breakage-resistant boundary walls 36, 36′ (FIG. 5) of the two sub-dosage units 12,12′ (see also boundary wall portions 36d,36d′ in FIG. 6). The bound powder matrix of the functional seam has a lower break strength than the adjoining boundary walls, to provide improved subdivision of the tablet into the two (or more) sub-dosage units 12,12′. Each of the two (or more) sub-dosage units 12,12′ includes a body that includes respectively the planar breakage-resistant boundary wall 36,36′, an outer peripheral wall 38,38′, the top wall 16,16′, and the base wall 17,17′. The functional seam 20 extends axially to bisect both a base bound-powder layer 13 and a top bound-powder layer 14 and extends laterally to sidewall portions 21 that bisect the outer peripheral wall 38,38′ of the sub-dosage units 12,12′ of the tablet 10.

The dividable tablet 10 comprises, and typically consists of, a plurality of bound-powder layers, including the one or more base bound-powder layer 13, the one or more top bound-powder layer 14, and a plurality of intermediate bound-powder layers 15, which in the illustrated embodiment of FIG. 5 includes six intermediate bound-powder layers 15a through 15f. FIG. 6 shows the dividable tablet 10 as a series of incremental, bonded layers of bound powder.

The one or more base bound-powder layer 13 (shown in FIG. 6) comprises a base functional seam portion 22b that extends to the peripheries 21b to join or bond together the pair of opposed base wall portions 17,17′ including the base boundary wall portion 35b,35b′ that join or bond along opposite sides of the base functional seam portion 22b.

The top bound-powder layer 14 (shown in FIG. 5) comprises a top functional seam portion 22t that extends to the sidewall portion 21t to join or bond together the pair of opposed top wall portions 16,16′ including top boundary wall portion 35t,35t′ joined or bonded along opposite sides of the top functional seam portion 22t.

Each of the plurality of intermediate bound-powder layers 15 (referred to collectively as layers 15x, and individually as layers 15a-15f) comprises respectively an intermediate functional seam layer portion 20 (referred to collectively as portions 20x, and individually as portions 20a-20f), and opposed intermediate sub-layer portions 18 and 18′ (referred to collectively as layer portions 18x, and individually as layer portions 18a-18f) joined or bonded along opposite sides of the intermediate functional seam layer portions 20x.

The top of the fourth intermediate layer 15d shows the intermediate functional seam layer portion 20d joined to and separating two half-layers identified as the intermediate sub-layer portions 18d,18d′. Each intermediate sub-layer portion 18d,18d′ consists of a boundary wall portion 36d,36d′, a peripheral wall portion 38d,38d′, and an intermediate interior portion 30d,30d′. The other five intermediate layers 15a-15c and 15e-15f each have the same construction as the fourth intermediate layer 15d, though can be different.

The functional seam 20 of the dividable tablet 10 therefore consists of a series of incremental bound-powder layers that include the base functional seam portion (22b, FIG. 6), the plurality of intermediate functional seam portions (20a-20f, FIG. 6), and the top functional seam portion 22t (FIG. 5), stacked vertically and bound to one another. Likewise, the boundary walls 36,36′ consist respectively of the base boundary wall portions 35b,35b′ of the base layer 13, the intermediate boundary wall portions 36x,36x′, and the top boundary wall portion 35t,35t′, stacked vertically and bound to one another.

The sub-dosage unit 12 (and the opposed sub-dosage unit 12′ respectively) shown in FIG. 5 consists of the planar, breakage-resistant boundary wall 36, hereinafter referred to just as “boundary wall”, the peripheral wall 38, base wall 17, top wall 16, and interior portions 30x.

The boundary wall 36 consists of a series of layers that include the base boundary wall portion 35b of the base layer 13 shown in FIG. 6, the intermediate boundary wall portions 36a-36f of the intermediate layers 15x, such as intermediate boundary wall portion 36d of intermediate sub layer 15d shown in FIG. 6, and the top boundary wall portion 35t of the top layer 14 shown in FIG. 5, which are joined together during the 3D printing process.

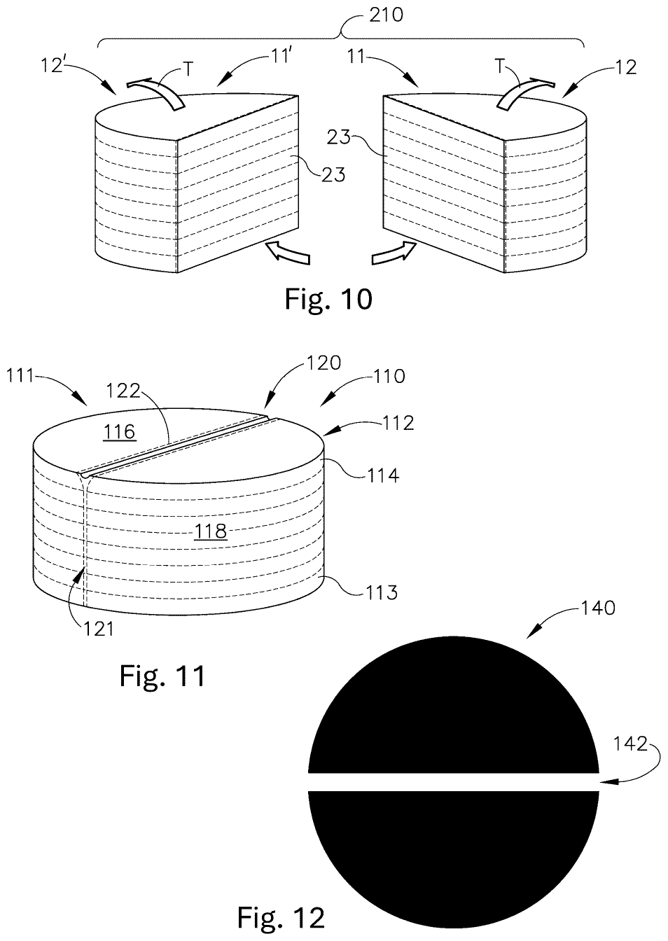

The peripheral wall 38 consists of the peripheral wall portions 38x of the intermediate bound-powder layers 15x (see FIG. 6). The peripheral wall 38, the top wall 16, and the base wall 17, with the respective boundary wall 36, form a unitary outer shell of each sub-dosage unit 12. The peripheral wall 38, the top wall 16, and the base wall 17 components of the outer shell independently comprise a third bound powder matrix that has a third break strength, which is typically greater than the break strength of the bound powder matrix of the functional seam 20, to ensure that both the boundary walls 36,36′ and the sub-dosages units 12,12′ do not crack or break when the tablet 10 is divided along the functional seam 20 into the two separate sub-dosage halves 11,12 (see FIG. 10).

The intermediate interior portions 30x,30x′ of the tablet 10 can comprise a bound powder matrix or an unbound powder. In various embodiments, the intermediate interior portion 30x,30x′ of the tablet 10 comprise a bound powder matrix having a break strength that neither contributes to nor diminishes the breaking of the tablet into sub-dosage units. As an increase in a break strength of a portion of the bound powder matrix can increase with binder content, and decrease possibly the orodispersive quality and/or disintegration rate of such portion of the sub-dosage units, it can be preferred to minimize the binder content, and therefore the break strength, of the intermediate interior portions 30x,30x′ of the tablet 10, to ensure target disintegration rate while minimizing and avoiding breakage of the sub-dosage units when dividing the tablet along the functional seam 20.

Preferably the two or more sub-dosage units 12,12′ have equal mass and ingredient content after dividing. Forming of the functional seam 20 between the boundary walls 36, 36′, as shown in FIGS. 5 and 6, can be done using a similar process as described above for forming the conventional dosage forms shown in FIGS. 1 and 2, by inclusion of a printing gap 42 in the liquid printing pattern in each layer along a bisecting line 101. The printing gap 42 is typically a portion of the printing pattern where no binding liquid is applied.

Forming a dividable tablet 10 includes forming a plurality of bound-together layers of a build powder. FIG. 6 shows a base layer 13 of bound powder, formed by printing a layer of build powder material with a binding liquid, thereby bonding the powder material in the printed areas into a bound-powder layer. A printing nozzle prints a multiplicity of droplets accurately according to a pre-determined (designed) printing pattern, with high precision and resolution within selected areas or regions of the surface of the powder layer, and at a volumetric or mass rate sufficient to wet the particles of powder material in the printed areas of the powder layer.

In the illustrated embodiment, the printing pattern 40 used to form the base layer of bound powder is illustrated in FIG. 8. The two dark (and darkest) areas are in the shape of semi-circles 41 and 41′, including chord edges 43,43′. The dark filling of the dark area indicates that the binding liquid applied by the print nozzles, under the control of a printing program and controller, is 100% of a pre-determined saturation level. In some embodiments, the 100% saturation level corresponds to a quantity of binding liquid of typically about 25 to about 35 mg per square centimeter and per millimeter thickness (mg/cm²/mm) of the build powder layer.

In other area(s) or region(s) within the dosage form, such as, in a non-limiting example, semi-circular print areas 48,48′ of the printing patterns 45,45′ in FIG. 9, binding liquid can be applied to interior portion(s) 30x of intermediate bound-powder layers 15x at a saturation level less than the 100% saturation level applied to semi-circles 41 and 41′ and the chord edges 43,43′ of base layer 13, but also greater than the 0% saturation level of the printing gap 42. In some embodiments, the quantity of binding liquid is about 6 to 25 mg/cm²/mm, more typically about 10 to about 25 mg/cm²/mm of the build powder layer.

Without being bound by a particular theory, it is believed that binding liquid applied onto a layer of powder material in these areas will migrate downward from the surface of the powder (where the liquid has been applied) through most or all of the thickness of the powder layer, thereby wetting most or substantially all of the powder material within the printed area. If a printed area(s) does not cover the entire upper surface of the powder layer, and there is one or more unprinted areas beyond the periphery of the printed area(s), the binding liquid deposited in the interior of the printed area will migrate downwardly (vertically) through powder beneath the printed area, while the binding liquid deposited at or near the periphery of the printed area will migrate both downwardly (by gravity and capillary action) through the powder beneath the periphery of the printed area and laterally (by capillary action) into the powder in the adjacent unprinted area. Once an upper surface of a powder layer has been wetted, and a next powder layer is deposited thereupon, it is believed that a portion of residual liquid at the upper surface of the first layer can migrate upwardly (by capillary action) and into the underside of the next powder layer, which is believed to ensure a bonding of the two successive powder layers into a unitary bound-powder structure.

The opposed chord edges 43,43′ of the respective semi-circles 41,41′ are parallel and spaced apart by the printing gap 42, where either no droplets of the binding liquid are deposited, or only a minimal quantity of droplets are deposited. Within the incremental powder layers, a width of the printing gap 42 is selected, and a volumetric or mass concentration of the binding liquid is applied along the chord edges 43,43′ of the respective semi-circles 41,41′, to allow at least a minimum amount (volume or mass) of the printed binding liquid that has been applied along the chord edges 43,43′ to migrate laterally into the zone of powder within the printing gap where binding liquid has not been printed. Without being bound to any particular theory, the level of lateral migration of the binding liquid into and through the powder material, coupled with any solvent vapor exposure during the drying step, is sufficient to bind together the particles of the powder layer within the zone of the printing gap, providing bound powder in the zone of the printing gap with a break strength sufficient to bind together the at least two sub-dosage units into a tablet that remains bound together under ordinary handling conditions, until a user manually divides the tablet along the functional seam. Determining the precise amount of binding liquid that migrates laterally toward or into the unprinted zone of powder is impractical, and not readily determinable.

The width or lateral dimension of the functional seam 20 is typically about 100 μm to about 500 μm, though preferably about 200 μm to about 400 μm, although the precise boundaries between the confront surfaces of a boundary wall and the functional seam 20 can be difficult to determine accurately. In various embodiments, the narrower width of a functional seam is preferred, provided its break strength is sufficiently high to secure or connect together the two sub-dosage units of the tablet, in order to minimize the loss of powder along the functional seam when the tablet is divided, and to promote dividing the dividable tablet into two or more substantially equivalent sub-dosage units along the functional seam. However, the functional seam width must also be adequate to ensure the two dosage form sub-dosage units do not fuse together excessively in one or more regions of the functional seam, which might lead to variability in the effective fracture plane when dividing the dosage form as intended.

In various embodiments, after the dividable tablet has been divided, the divided sub-dosage units have equal mass with a difference between the masses of the two divided sub-dosage units being about 3% or less, which can be about 2% or less, 1.0% or less, and 0.5% or less, and the amount of powder material of the dividable tablet that is separated or lost after the dividable tablet has been divided is less than 3%, which can be less than 2%, less than 1.0%, less than 0.5%, less than 0.3%, and less than 0.1%, of the total mass of the undivided tablet.

In some embodiments, the width of the printing gap can be widened by printing or dispensing a grayscale saturation level of binding liquid within the zone of the printing gap. The grayscale saturation level of binding liquid would be expected to be less than about 50%, more typically at least 10%, and/or up to about 30%, and including about 10% to about 25%. While effective in forming a dividable tablet, the broadening of the width of the printing gap to include a grayscale printing pattern may result in the bound powder matrix within the functional seam being less tenacious, resulting in the spalling (or, falling away) of particles of the powder material from and along the functional seam.

Without being bound by any particular theory, the relative saturation level of binding liquid within the powder disposed in the zone of the printing gap, to form the functional seam, is about 10% to about 75%, more particularly about 10% to about 25% saturation. Typically, the saturation level of the bound powder matrix of the functional seam is uniform through the entire span of the functional seam.

A preferred embodiment provides a dividable tablet that divides into two (or more) sub-dosage units of equal weight and dosage amount, and with minimal particle loss along the functional seam. It is believed that a method that minimizes the printing gap optimizes the seam-breaking and tablet-dividing results.

FIG. 6 illustrates the manufacturing of a dividable tablet as an incremental series of intermediate layers of bound powder formed by printing of the intermediate layers of powder material with a binding liquid using a printing nozzle that prints a multiplicity of droplets accurately according to a printing pattern, with high precision and resolution at selected areas or regions of the surface of the powder layer, and at a selected volumetric or mass rate sufficient to wet the particles of powder material in the printed areas of the powder layer, thereby bonding the powder material in the printed areas into the intermediate bound-powder layers. In the illustrated embodiment, six intermediate bound-powder layers (15a-15f) have been formed using the same printing pattern 44 shown in FIG. 9. The printing pattern 44 includes two opposed, semi-circular printing patterns 45,45′. Each of the semi-circular printing patterns 45,45′ respectively includes a peripheral print area that includes an outer peripheral print area (47,47′ respectively) and a chord edge print area 46,46′. The dark areas, as described above, indicate that the binding liquid is applied by the print nozzles, under the control of a printing program and controller, preferably at a solid printing pattern, to deliver a 100% saturation level of binding liquid.

In the interior, semi-circular print areas 48,48′ of the printing patterns 45,45′, any quantity of binding liquid can be applied, or no binding liquid is applied, to the powder layers. The powder within these interior portions 30x,30x′ of the intermediate layers are entirely enclosed within a surrounding or enveloping wall of a strongly bound powder matrix, namely, the top wall 16, the base wall 17, the boundary wall portions 36,36′, and the peripheral wall portions 38,38′, which allows the powder within these interior portions 30x,30x′ to be unprinted, printed with a grayscale print pattern (less than 100% saturation), or printed with a solid printing pattern. Preferably, interior portions 30x,30x′ can be printed at a saturation level of about 20% to about 40%, and more preferably about 25% to about 35%.

In a non-limiting example, an intermediate bound-powder layer 15x can be printed according to printing pattern 45 of FIG. 9 at a 30% grayscale printing level relative to a base layer 13 and top layer 14 printed according to printing pattern 40 of FIG. 8 at a solid printing pattern. The intermediate bound-layer 15x can have a liquid-to-powder weight ratio of about 0.10 to about 0.20, preferably about 0.13 to about 0.17, and a mass saturation of about 6.2 mg/cm²/mm to about 12.3 mg/cm²/mm, preferably about 8.0 mg/cm²/mm to about 10.4 mg/cm²/mm. Accordingly, the resulting tablet can have an average liquid-to-powder ratio of about 0.25 to about 0.45, preferably about 0.30 to about 0.40, and a mass saturation of about 15 mg/cm²/mm to about 28 mg/cm²/mm, preferably about 18 mg/cm²/mm to about 25 mg/cm²/mm.

Again, without being bound by any particular theory, when printed with grayscale or 100% (full) saturation level, it is believed that the binding liquid applied onto a layer of powder material in these areas can migrate downward from the surface of the powder where the liquid has been applied, and through the thickness of the powder layer, thereby wetting some, most, or all of the powder material in these printed areas.

Typically, after a suitable threshold level for binding is achieved, it is preferred to minimize the amount of binding liquid applied to a powder layer, if only to avoid having to evaporate or dry excessive amounts of solvents that remain after the forming of the bound powder matrices. In such situations, the break strength of the bound powder matrix within the interior contained areas 30,31, formed by the semi-circular printing areas 48,48′, is lower than the break strength of the bound powder matrix that forms the opposing boundary walls 36, 36′, and lower than the break strength of the bound powder matrix that forms the peripheral wall portions 38,38′, top wall 16, and base wall 17.

To complete the dividable tablet 10, as shown in FIG. 5 and FIG. 6, one or more top layers of bound powder is formed by printing a layer of powder material with a binding liquid using a printing nozzle that prints a multiplicity of droplets accurately according to a printing pattern, with high precision and resolution at selected areas or regions of the surface of the powder layer, and at a volumetric or mass rate sufficient to wet the particles of powder material in the printed areas of the powder layer, thereby bonding the powder material in the printed areas into a bound-powder layer. In the illustrated embodiment, the printing pattern 40, shown in FIG. 8, which was used to print the base layer 13, is used to print the top layer 14 of powder to form the top wall 16 of bound powder. The two dark areas are in the shape of semi-circles 41 and 41′, including chord edges 43,43′. The dark filling of the dark area indicates that the binding liquid is applied by the print nozzles, under the control of a printing program and controller, preferably at a full saturation level. The binding liquid that is applied onto a layer of powder material in these areas will migrate downward from the surface of the powder (where the binding liquid has been applied) through the thickness of the powder layer, thereby wetting most or substantially all of the powder material within and beneath the printed area. Typically, binding liquid will migrate through most of or the entire thickness of the printed powder layer. Typically, the width of the printing gap 42 of the top layer printing pattern 40 is the same or similar to that of the intermediate layer printing pattern 44, and of the pattern used for printing the base layer 13.

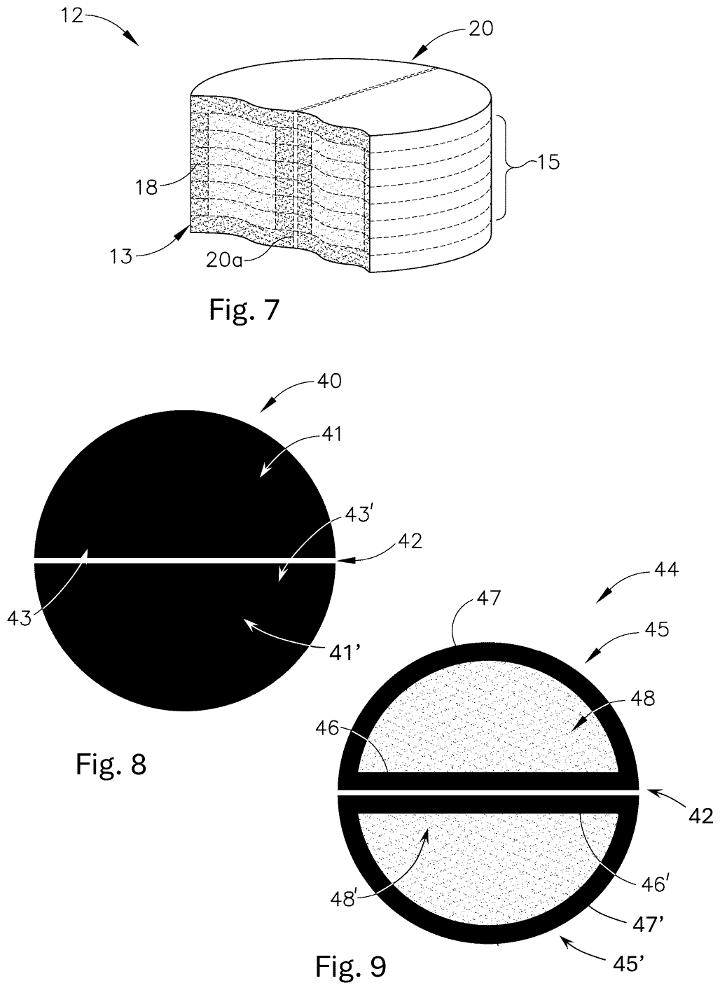

FIG. 7 is a perspective view of the tablet with an irregular segment removed from the sub-dosage units 12 and 12′ along and perpendicular to the functional seam 20, to reveal the interior portion of each sub-dosage unit 12 and 12′.

Later, after any excess binding liquid has been evaporated (naturally or by a drying process) from the wetted powder, a stable, bound powder matrix results. The bound powder matrix in the areas where a programmed 100% or full saturation of the binding liquid has been applied will have a break strength that will resist breakage of the bound powder matrix in such areas under ordinary handling conditions of the tablet by users.

### Porosity

For a given build powder composition and binding liquid composition, the saturation level of the binding liquid correlates with the break strength of the resulting porous bound powder matrix, and inversely with the porosity of the bound powder matrix. Consequently, an estimate of relative porosity of a bound powder matrix or portion thereof can be used to predict or estimate the relative break strength of the powder matrix or the portion thereof.

The bound powder matrix includes bound powder matrix portions of a functional seam 20, a boundary wall(s) 36, a peripheral wall 38, a base wall 17, a top wall 16, and an interior portion(s) 30x, each individually having a porosity from about 20% to about 90%. The boundary walls 36 have a porosity of about 25% to about 90%, which can be at least 40%, or at least 50% and up to 70%. The functional seam 20 has a porosity of about 20% to about 50%, which can be at least 25%, or 35%, and up to about 50%. The porosity of the functional seam 20 is higher than the porosity of the boundary walls 36, and is at least 5% higher, which can be at least 10%, or at least 15%, or at least 20% higher, or up to about 50% higher, which can be up to 40%, or up to 30%, or up to 40% higher, than the porosity of the boundary walls 36; for example, about 15% to about 25% higher. Without being bound by any particular theory, the higher porosity of the bound powder matrix portion of the functional seam, as compared to that of the boundary wall, results in a reduced break strength of the bound powder matrix portion of the functional seam, as compared to the break strength of the boundary walls. Consequently, the higher break strength of the bound powder matrix portion of the opposed boundary walls causes the bound powder matrix of the functional seam to fail and break when a bending force is applied to the dividable tablet across the functional seam, dividing the tablet into the two sub-dosage units.

The peripheral wall 38 has a porosity of about 20% to about 70%, which can be at least 30%, or at last 40% and up to 60%. The porosity of the peripheral wall 38 is lower than the porosity of the functional seam 20, and can be at least 5% lower, and more typically at least 10%, or at least 20%, or at least 30%, lower than the porosity of the functional seam 20. The peripheral wall 38 typically as a porosity that, excepting the boundary walls, is the same or lower than the other portions of the bound powder matrix, to maintain the integrity of the tablet sub-dosage units before and after division under ordinary handling conditions. The porosity of the periphery can be the same, or lower, or higher than, the porosity of the boundary walls.

In various embodiments, the break strength, or hardness, of a bound powder matrix is proportional to the concentration of a binder material and/or binding liquid within the bound powder matrix. The content of binder material within the bound powder matrix can be introduced within the powder material that is deposited and layered, or within the binding liquid composition, or both. Other factors can also contribute to the break strength, including though not limited to the particle size distribution of the powder material, the powder composition, and the print image.

Although the illustration of the dividable tablet 10 shown in FIG. 5 uses certain light, dashed lines to illustrate the borders of different bound-powder portions of the bound-powder layers, the visual appearance of the outer surfaces of an actual dividable tablet will appear approximately uniform. For example, the zones of a bound powder matrix that have been printed with a 100% saturation and with a 50% saturation can, and often do, appear the same or similar to the naked eye after excess solvent has been evaporated away.

FIG. 10 illustrates a dividable tablet 210 that has been divided along the functional seam such as seam 20 illustrated in FIG. 5, into two substantially equal sub-dosage halves 11,11′. The tablet 210 can be divided along the functional seam 20 by grasping the sub-dosage units 12,12′ of the tablet 210 between the fingers on opposite sides of the functional seam 20 and applying opposing forces (arrows marked “T”) to the sub-dosage units 12,12′ until the dividing functional scam 20 breaks. The subdivided tablet 210 consisting of the resulting tablet halves 11,11′ will typically include some minimum residue 23 of the functional seam 20 attached to each of the sub-dosage units 12,12′, and a minimum amount of lost powder that may flake off and fall away due to dividing.

FIG. 11 illustrates another embodiment of the invention wherein a physical score line is formed along the surface of the top layer 114, or the base layer 113, or both, with the physical score line extending in the same plane and parallel with the functional seam 20. The physical score line 122 assists the user in identifying the position and orientation of the functional seam 20. In a typical embodiment, the appearance of the bound powder matrix at the top surface of the sub-dosage units may appear identical to, or indistinguishable from, the appearance of the bound powder matrix at the top surface of the functional seam, and the user may not be able to see or discern the functional seam, and may be uncertain of where to grasp the tablet 10 correctly for dividing. In the illustrated embodiment, the top layer 114 of bound powder is formed using a printing pattern 140 for the binding liquid as shown in FIG. 12, which has a wider printing gap 142 as compared to the printing gap 42 (shown in FIG. 9) used in the printing of the intermediate layers 15. In some embodiments, one or more of the uppermost intermediate layers 15 may also be printed with a wider printing gap, to ensure that a row of unbound powder material is left unwetted by the printed binding liquid, which when removed leaves a shallow, physical line, illustrated as a gulley 122, which extends along the functional seam 120 and bisects the top layers 11 of the tablet 110.

### Dosage Form Characterization

The following procedures are used to characterize any of the tablets described herein.

### Surface Texture

The tablets are inspected visually with or without the aid of a microscope. The texture of the tablets is generally analyzed to determine if the surface is rough or smooth and whether the edges on the upper surface and edges of the tablet perimeter are clean and sharp or rough and jagged.

### Hardness

Tablets are analyzed for overall hardness as determined by a tablet breaking force assay according to USP <127>using a VK 200 tablet hardness tester (Varian, US). The strength or hardness of the tablets is measured by a fracture test. A tablet is centered between the jaws of the tester and force is applied until the tablet fractures. The load at fracture is returned in kiloponds (kp). A kilopond is a metric unit of force measurement with 1 kp being equivalent to 9.807 Newtons.

Dispersion time

Tablets are analyzed for disintegration time using a basket-rack assembly according to the procedure described in the USP <701>.

### Bulk Density

The bulk density of the tablet is determined by measuring the tablet's mass and dividing that value by the calculated volume of the tablet. The tablet volume is calculated by measuring its dimensions and using the proper mathematical formula according to the tablet shape. For example, for cylindrical tablets, the volume is calculated using the geometric formula, V=πr²h wherein r is the radius of the tablet and h is its height. Typically, a tablet weighing 320 mg, and having a height and diameter of 5.15 mm and 11.6 mm, respectively, has a volume of about 0.544 cm³ and a bulk density of 0.59 g/cm³.

### Porosity

The tablet's porosity is calculated by determining the volume of the solids of the particulate material within a tablet compared to the volume of the intact tablet volume. A tablet is pulverized into a fine particulate material and highly compacted, and the volume of the pulverized, compacted particulate material is determined. Porosity is determined according to Formula I, below:

$$porosity = \frac{intact volume - particulate volume}{intact volume} * 100$$

As a non-limiting example, a tablet weighing 320 mg, and having a height and diameter of 5.15 mm and 11.6 mm, respectively, is determined to have an intact volume of 0.544 cm³ and a particulate volume of 0.211 cm³. Accordingly, the porosity of the tablet is approximately 61%.

Alternatively, the porosity of a tablet can be determined using mercury porosimetry, in which the porosity of a material is measured by applying controlled pressure to a sample immersed in mercury, which cannot otherwise penetrate pores because of its high contact angle. Volume and pore size distributions of the material can be determined from the amount of pressure required to intrude into the pores and analyzed using the Washburn equation. It is expected that a tablet's porosity determined by mercury porosimetry will coincide with the porosity calculated above based on the intact and pulverized tablet volumes.

### Analysis of Divisible Tablet Using X-ray Computed Tomography

The present invention also provides a method detecting the porosity of a bound powder matrix employing X-ray computed tomography. The technique provides taking a series of sectional scans through any of the orthogonal dimensions of the tablet. The X-ray scan detects a density of a multiplicity of voxels of sub-micron-or micron-size in each scanned section, to detect the true density of the voxel. If a portion of a particulate of a compound is scanned, the true density of the compound is detected, and if a void space is scanned, a density of zero is detected. The CT technique then builds the series of scans into a three-dimensional model. Software associated with the scanning system can compute the average porosity for user-selected sections of the three-dimensional model.

A placebo tablet having a functional seam is made substantially in accordance with the method of Example 1, replace part-for-part the API compound (in Example 1, droxidopa) in a formulation with additional particulate mannitol. To detect the inner particulate mass and porosity patterns within the tablet, the placebo tablet was analyzed using computed tomography (CT) scanning. CT scanning combines a series of X-ray images taken from different angles around a tablet body and uses computer processing to create cross-sectional images (slices) of the bound powder matrix of the tablet. The CT scan images are generated from the detected voxels in the three-dimensional space of the tablet. In the scanned images, higher gray-scale voxels represent a higher density (or more radio-opaque) voxel value.

The dividable tablet is made having two sub-dosage units separated by a functional seam, each sub-dosage having a boundary wall, an outer peripheral wall and an interior portion, similar to that illustrated in FIGS. 5-7. The dividable tablet is made from a series of incremental bound-powder layers, including one or more bottom layers, one or more top layers, and a plurality of intermediate layers therebetween. The top and bottom bound-powder layers are made with a printing pattern as shown in FIG. 8 using a 100% binding liquid print saturation, and the intermediate bound-powder layers are made with a printing pattern as shown in FIG. 9, using a 100% binding liquid print saturation for the boundary print pattern and peripheral print pattern, and a 30% binding liquid print saturation for the interior print pattern.

The porosity of the dividable tablet was scanned and analyzed using x-ray computed tomography to make an estimate of the porosity of the bound powder matrix of the respective portions of the tablet, including the functional seam, the two boundary walls, the peripheral, top and bottom walls, and the interior portion. Scans were taken laterally through the height of the tablet, parallel with the incremental bound-powder layers.

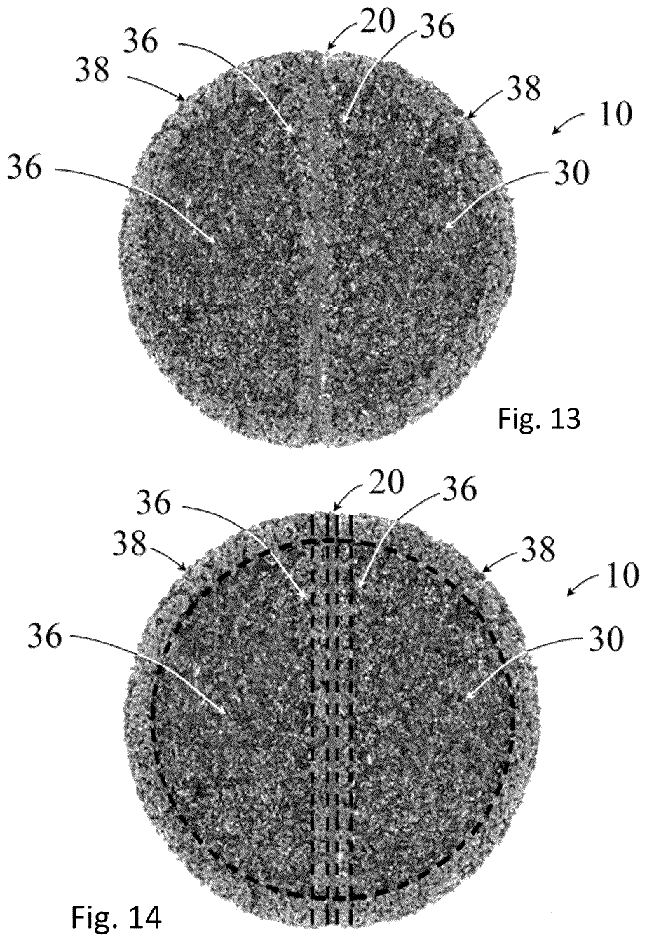

FIG. 13 illustrates a plan view of a CT scan taken laterally through the height of the dividable tablet, parallel with the incremental bound-powder layers. FIG. 14 shows the CT of FIG. 13, with the boundaries between the functional seam, boundary walls, peripheral walls, and interior portions illustrated in dashed lines to better distinguish such elements.

The CT scanning procedure involves adjusting a variety of scanning parameters in order to detect and calculate a density for each voxel (volumetric scanning unit) of each scanned layer of the tablet. The procedures and results shown for the determined porosity of each scanned portion of the tablet are understood to be relative porosities, rather than absolute or true porosities.

The relative porosities of the scanned tablet, and the ratio of the functional seam porosity to the barrier walls and peripheral walls, are listed below:

| | | |
|---|---|---|
| Tablet average | 14.3% | — |
| Functional seam | 17.7% | 1.0 |
| Boundary walls | 14.5% | 0.82 |
| Interior of sub-dosages | 15.3% | 0.86 |
| Peripheral walls | 11.2% | 0.63 |

As shown, the measured porosity of the functional seam is about 22% higher than the measured porosity of the boundary walls, and about 59% higher than the measured porosity of the peripheral walls. As a consequence, the breaking force of the bound powder within the functional seam will be significantly less than that of the boundary walls and peripheral walls, resulting in preferential breakage along the more-porous functional seam.

### EXAMPLES

### Resistance to Breaking Testing

A test of the resistance of a dividable tablet to a breaking force can use a three-point bending test along a centerline (or other lateral portion) of the tablet. A description of three-point testing of articles is described as https://textureanalysisprofessionals.blogspot.com/2018/10/three-point-bend-testing-using-texture.html, the disclosure of which is incorporated by reference.

A test setup provided herein for detecting that a functional seam has a lower break strength than the boundary wall portions of the dividable tablet uses a human tester's fingers as the break force device, and applies the opposed break forces to the opposed lateral sides of the tablet that is placed over a linear fulcrum, such as a wire of a #1 paper clip (0.033 wire gauge), on a test surface. A break force is measured, or perceived, by aligning the lateral centerline of the tablet over the linear fulcrum, so that the dividable tablet balances on, or rests with one side of the tablet touching the test surface. Gently, the tester places a forefinger tip of both hands onto the opposed lateral sides of the tablet, and presses the fingers downwardly, normal to the test surface, until the tablet breaks into two (or more) sub-dosage units.

When testing the break strength of the functional seam, the functional seam of the tablet is aligned with, and placed on top of, the linear fulcrum. The resistance of the tablet to a break force with the functional seam aligned over the linear fulcrum will be slight, and the dividable tablet breaks into two sub-dosage units with little applied force.

When testing the break strength of the boundary walls, both the functional seam and the boundary walls of the tablet are aligned transverse to (at 90 degrees with) the linear fulcrum. The resistance of the tablet to a break force with the boundary walls transverse to the linear fulcrum will be more significant, and the dividable tablet does not break into smaller units with a little (or slightly more) applied force, and only breaks when a significant downward force is applied, typically along a rough break line into two or more different and unequal sub-dosage units.

Example 1: An open bed apparatus was used to prepare dosage forms, similar to one described in U.S. Pat. No. 8,888,480, the disclosure of which is incorporated by reference in its entirety. Multi-layer rapidly orodispersible dosage forms were prepared into the form of dividable tablets having two API active strengths: 400 mg and 600 mg. The powder material and the printing liquid contained the following components shown in Table A and B. The composition of the three tablets is shown in Table C.

TABLE A
Powder material

| Ingredient | Trade name | Concentration % wt/wt total |
|---|---|---|
| Droxidopa | Milled | 35.0 |
| Mannitol, USP | Pearlitol 160C | 47.9 |
| Microcrystalline | Avicel PH101 | 12.0 |
| cellulose | | |
| Hydroxypropyl | HPC-L-FP | 4.60 |
| Cellulose | | |
| Colloidal silicon | Cab-O-Sil M5P | 0.50 |
| Dioxide, NF | | |

TABLE B
Binding Liquid

| | | Concentration % wt/ |
|---|---|---|
| Ingredient | Trade name | wt total |
| Purified water, USP | N/A | 73.0 |
| Isopropyl alcohol, USP | N/A | 12.3 |
| Copovidone, NF | Kollidon VA 64 | 10.5 |
| Glycerin, USP | N/A | 1.90 |
| Polysorbate 20, NF | N/A | 1.90 |
| Anhydrous citric acid, USP | Powder grade | 0.400 |

TABLE C
Tablet Composition

| | Trade | | 100 mg | | 400 mg | | 600 mg | |
|---|---|---|---|---|---|---|---|---|
| Component | name/grade | Manufacturer | mg | % w/w | mg | % w/w | mg | % w/w |
| Droxidopa | Milled | MSN | 100 | 33.0 | 400 | 33.3 | 600 | 33.3 |
| Mannitol, USP | Pearlitol | Roquette | 137 | 45.2 | 547 | 45.6 | 821 | 45.6 |
| | 160C | | | | | | | |
| Microcrystalline | Avicel PH | FMC/- | 34.3 | 11.3 | 137 | 11.4 | 206 | 11.4 |
| cellulose, NF | 101 | Dupont | | | | | | |
| Hydroxypropyl | HPC-L-FP | Nisso | 13.1 | 4.34 | 52.6 | 4.38 | 78.9 | 4.38 |
| cellulose, NF | | (Nippon | | | | | | |
| | | Soda) | | | | | | |
| Colloidal | Cab-O-Sil | Cabot | 1.43 | 0.472 | 5.71 | 0.476 | 8.57 | 0.476 |
| silicon dioxide, | M5P | | | | | | | |
| NF | | | | | | | | |
| Copovidone, | Kollidon | BASF | 12.1 | 4.01 | 41.9 | 3.49 | 62.1 | 3.45 |
| NF | VA64 | | | | | | | |
| Glycerin, USP | 100% | Avantor | 2.20 | 0.725 | 7.59 | 0.631 | 11.2 | 0.624 |
| | vegetable | | | | | | | |
| | base | | | | | | | |
| Polysorbate 20, | N/A | Penta | 2.20 | 0.725 | 7.59 | 0.631 | 11.2 | 0.624 |
| NF | | | | | | | | |
| Anhydrous | Powder grade | JT Baker | 0.462 | 0.153 | 1.60 | 0.133 | 2.37 | 0.131 |
| citric acid, USP | | | | | | | | |

| | | | | | | |
|---|---|---|---|---|---|---|
| Total | 303 | 100 | 1202 | 100 | 1801 | 100 |

Each of the multiple layers of the powder material deposited as a bed of powder had a uniform thickness of about 400 micrometers. After each powder layer was deposited, the bed of powder was passed under a bank of liquid printing nozzles (Starfire 1024 LA, from Fuji Dimatix) to print a pattern of binding liquid onto the surface of the powder layer in a prescribed printing pattern, at a precise location for each dosage form.

For the 600 mg dosage tablet, first and second layers of powder material were deposited, and each was sprayed in sequence with a printing pattern 40 as illustrated in FIG. 8, consisting of saturation level of 31 mg binding liquid per cm² of area and millimeter (mm) depth of the powder layer (mg/cm²/mm), defining a 100% saturation level, in two semi-circular patterns 41,41′ that share a diameter of 23.4 mm, and are bisected or divided by a 0.25 mm wide printing gap 42 where no binding liquid is printed. Next, eight additional layers of powder material were deposited, each sprayed with a printing pattern 44 as illustrated in FIG. 9, consisting of two semi-circular patterns 45,45′ that each have a respective peripheral pattern 47,47′ and respective boundary wall patterns 46,46′ of 100% saturation level of binding liquid, and respective interior portions 48,48′ of a 30% grayscale saturation level. The two semi-circular patterns 45,45′ were separated by the same 0.25 mm wide printing gap between the respective boundary wall patterns 46,46′, and registered over the printing gap of the previous powder layer. Binding liquid dispensed in the boundary wall patterns formed respective boundary walls consisting of a bound powder matrix. Last, two final layers of powder material were deposited and each sprayed separately with the same printing pattern 40 as illustrated in FIG. 8, consisting again of 100% of saturation level of binding liquid in two semi-circular patterns 41,41′ with the 0.25 mm-wide printing gap 42, and again registered over the printing gap of the previous powder layer. Portions of the binding liquid that were printed on both sides of the printing gap within the boundary wall patterns 46,46′ (at 100% saturation level) migrated into the printing gap zone of the powder layer, to at least partially wet or dampen the powder material and form a functional seam consisting of a bound powder matrix that was bound with the bound powder matrices of the respective boundary walls. The bound powder matrix of the functional seam was sufficiently strong and tenacious to form a unitary bound-powder tablet that retained its form under ordinary handling conditions, though was weaker and less tenacious than the bound powder matrices of the boundary walls of the two sub-dosage units, which allowed a user to break the finished tablet along the functional seam, with minimal loss of powder and substantially equal masses in the two sub-units.

The second dosage form of 400 mg was formed using a printing pattern diameter of 20.4 mm.

### Example 2

### Functional Seam With Physical Score Line

Using the same apparatus and process as for Example 1, a dividable tablet with a physical score line was made. All steps were the same except for the printing of the last two powder layers. After each of the two final layers of powder material were deposited, the binding liquid was sprayed with a printing pattern 140 as illustrated in FIG. 12. The wider printing gap 142 resulted in a center portion for the powder material of the top powder layers within the zone of the printing gap 142, from being wetted and/or bound by the binding liquid. After the printing of the two top layers, the unbound powder material within the zone was removed, leaving a gulley in the top surface of the dividable tablet that is easily physical to indicate to a user where the tablet should be divided into two functionally equivalent tablet halves.

While particular embodiments of the invention have been illustrated and described herein, such details are not intended to restrict or limit the scope of the appended claims. Accordingly, while some embodiments are particularly described and illustrated herein, it should be understood that additional modifications and variations of these embodiments, and the equivalents thereof, are within the scope of the invention as recited in the following claims.

We claim:

1. A dividable tablet comprising a bound-powder matrix, the bound-powder matrix including at least two sub-dosage units and at least one functional seam, each of the at least two sub-dosage units including a boundary wall having a high break strength, wherein the boundary walls of the at least two sub-dosage units confront one another, and are joined along opposite sides of and to the functional seam, wherein the functional seam has a break strength that is less than a break strength of the boundary walls.

2. The dividable tablet according to claim 1, wherein the functional seam is configured to subdivide the sub-dosage units with the same mass within a mass tolerance of about 3% or less, and typically of 1.0% or less.

3. The dividable tablet according to claim 1, the functional seam is configured to subdivide the sub-dosage units with an amount of lost material that separates completely from the divided sub-dosage units after division of the tablet along the functional seam, wherein the amount of lost material is less than 3%, and typically less than 1.0%, and more typically less than 0.1% of the mass of the undivided dividable tablet.

4. The dividable tablet according to claim 1, wherein the at least one functional seam extends along a dissecting line that subdivides the dividable tablet into the two or more sub-dosage units, each sub-dosage unit including a breakage-resistant boundary wall joined along opposite sides of and to the one or more functional seams.

5. The dividable tablet according to claim 4, wherein the boundary walls comprise planar boundary walls, and each of the at least two sub-dosage units comprises a body including the planar boundary wall, wherein the planar boundary walls are oriented in parallel and confront one another, and the functional seam is positioned between and joined to the confronting planar boundary walls.

6. A dividable tablet, comprising:
(a) at least two sub-dosage units, each of the at least two sub-dosage units comprising a body comprising an active pharmaceutical ingredient (API), the body including a planar boundary wall, a periphery, and an interior body portion; and
(b) a functional seam, preferably through the center of the dividable tablet, that, when divided, divides the dividable tablet into the at least two sub-dosage units, the functional seam comprising a first bound powder matrix extending between the planar boundary walls of the at least two sub-dosage units,
wherein the first bound powder matrix has a first break strength, the respective planar boundary walls comprising a second bound powder matrix having a second break strength, the second break strength being higher than the first break strength, and wherein each planar boundary wall resists breakage of the at least two sub-dosage units when the dividable tablet is divided along the functional seam.

7. The dividable tablet according to claim 6, wherein the functional seam extends along a dissecting line of the dividable tablet, and extends through the entire structure of the dividable tablet to space apart completely the first sub-dosage unit from the second sub-dosage unit.

8. The dividable tablet according to claim 6, wherein the functional seam comprises a first bound powder matrix, and the boundary wall of each sub-dosage unit comprises a second bound powder matrix, the first bound powder matrix has a porosity that is at least 10% higher than a porosity of the second bound powder matrix.

9. The dividable tablet according to claim 8, wherein the higher porosity of the first bound powder matrix extends at least 50%, and typically up to 100%, of the length of the functional seam.

10. The dividable tablet according to claim 6, wherein the dividable tablet comprises from 20% to 80% by weight API, and provides a dosage amount of API of from 100 milligram (mg) to 2,000 mg.

11. The dividable tablet according to claim 6, wherein the bound-powder matrix comprises an active pharmaceutical ingredient (API), wherein the amount of the active pharmaceutical ingredient and can be subdivided along the functional seam so that each sub-dosage unit contains a sub-dose mass amount of the API that is a pre-determined fraction or portion of the total amount of API. In some embodiments, each sub-dose mass amount of the API is equal in mass.

12. The dividable tablet according to claim 11, wherein the dividable tablet does not include a physical score line in an outer surface of the dividable tablet, including along an axis of the functional seam.

13. The dividable tablet according to claim 12, wherein the dividable tablet includes a visible marking on or visible through the outer surface of the functional seam, to facilitate the identification of the functional seam and the positioning of the dividable tablet for sub-dividing along the functional seam.

14. The dividable tablet according to claim 6, wherein the dividable tablet can include a single functional seam through the center of the dividable tablet that, when divided, divides the dividable tablet into two equal sub-dosage units.

15. The dividable tablet according to claim 6, wherein the dividable tablet can be subdivided into three, four or more sub-dosage units.

16. A method of making a dividable tablet, comprising the steps of
i) providing a layer of a powder,
ii) printing a binding liquid onto the layer of powder using one or more dosage-form printing patterns for dispensing binding liquid onto the powder layer, wherein the dosage-form printing pattern includes at least two sub-dosage unit printing patterns, each sub-dosage unit printing pattern including a boundary wall portion for dispensing a boundary saturation of the binding liquid onto a boundary portion area of the powder layer to form a boundary bound powder matrix, wherein the boundary portions of the at least two sub-dosage unit printing patterns are confronting and separated by one or more printing gaps having a gap width, the one or more printing gaps having no saturation, or a reduced saturation as compared to the boundary saturation, of the binding liquid in a printing gap area of the powder layer, whereby a portion of the binding liquid dispensed in the respective boundary portion area of the powder layer migrates laterally into the printing gap area of the powder layer to provide a reduced saturation of binding liquid within the printing gap area of the powder layer that is sufficient to form the powder layer within the printing gap area forming a seam bound powder matrix that binds together the at least two boundary bound powder matrices, and
iii) repeating the steps i) and ii) one or more times, thereby forming the dividable tablet.

17. The method according to claim 16, wherein the printing gap has no binder saturation.

18. The method according to claim 17, wherein the gap width is at least 100 microns (μm), and up to 500 μm.

19. The method according to claim 16, wherein the boundary portion of the sub-dosage unit printing pattern has a width dimension, extending away laterally from the printing gap, of at least 100 μm.

20. The method according to claim 19, wherein the width dimension is at least about 300 μm.
